(19) United States
(12) Patent Application Publication
(10) Pub. No.: US 2002/0188013 A1
(43) Pub. Date: Dec. 12, 2002

(54) Composition for treatment of malignant tumors

(76) Inventor: Richard Frederick Dechant, Sacramento, CA (US)

Correspondence Address: RICHARD FREDERICK DECHANT, 500 DUNBARTON CIRCLE, SACRAMENTO, CA, 95825, US

(21) Appl. No.: 09/846,437

(22) Filed: May 2, 2001

(51) Int. Cl. A61K 31/426; A61K 31/125; A61K 31/05

(52) U.S. Cl. 514/365; 514/692; 514/731; 514/738

(57) ABSTRACT

A unique composition for treatment of malignant tumors and herpes virus infections is composed of resorcinol, camphor, and sodium sulfathiazole dissolved in propylene glycol. This composition is easy to apply, and at cocentrations used, causes no apparent side effects.

### REFERENCES

The Merck Index, Ninth Edition, Merck & Co., Rahway N.J., page 1058, No. 7951, 1976.

### TECHNICAL FIELD

The present invention is related to the chemical treatment of cancer. Malignant tumors sensitive to the composition are attacked via the blood stream and/or the lymph system, or by direct contact.

### BACKGROUND OF THE INVENTION

I originally formulated this composition to treat infections caused by streptococcus and staphlococcus bacteria. By chance I discovered it had properties for treating melanomas, carcinomas, internal malignant tumors, and vesicles caused by herpes simplex and zoster viruses, all of which are sensitive to this composition. When the composition is applied to the skin or to sensitive mucous membranes there are no deleterious side effects other than a slight 10 to 15 second stinging sensation.

### SUMMARY OF THE INVENTION

A composition to treat malignant tumors is composed of a mixture of resorcinol, camphor, and sodium sulfathiazole dissolved in propylene glycol. When the composition is applied directly to visible carcinomas, melanomas, and vesicles caused by herpes viruses, it causes these diseased tissues to dry up and heal within a few days. When the composition is absorbed through through subligual and rectal tissues it reaches susceptible internal malignant tumors and renders them inactive.

### EXAMPLE 1

| COMPONENT | % WT/WT |
|---|---|
| Resorcinol | 6.7 |
| Camphor | 5.0 |
| Sodium sulfathiazole | 2.1 |
| Propylene glycol | 86.2 |

6.7 grams of resorcinol, 5.0 grams of camphor, and 2.1 grams of sodium sulfathiazole are placed in a suitable container and allowed to stand for 4 to 5 hours so that a viscous liquid is formed. 86.2 grams of propylene glycol are added and thoroughly mixed so that all of the ingredients are completely dissolved. In these proportions, resorcinol and camphor blend together easily and dissolve rapidly in propylene glycol.

The example is given solely for the purpose of illustration and is not to be construed as a limitatio of the composition which may have many variations without departing from its spirit.

Method of Use;

The composition is applied to sublingual and rectal tissues by means of cotton swabs saturated with the composition. The swabs are allowed to remain in place from four to five minutes. Each swab holds about ¼ milliliter of the composition. For visible surface tumors, the composition is applied directly.

The composition differs from other anti-tumor compositions in that it requires no syntheses, no injections, and no oral ingestion. It begins action within hours and causes no apparent adverse side effects or skin irritation.

What is claimed is:

1. A composition for treatment of susceptible malignant tumors and herpes virus infections is prepared by dissolving 6.7 grams of resorcinol, 5.0 grams of camphor, and 2.1 grams of sodium sulfathiazole in 86.2 grams of propylene glycol.

2. The preparation according to claim 1. wherein the resorcinol remains at 6.7 grams, the camphor at 5.0 grams, but the sodium sulfathiazole is removed and the propylene glycol is increased to 88.3 grams.
